(19) United States
(12) Patent Application Publication
(10) Pub. No.: US 2006/0121566 A1
(43) Pub. Date: Jun. 8, 2006

(54) Stem cell expansion factor blocking gene limiting hox-induced expansion and method using such factor

(75) Inventors: Guy Sauvageau, Quebec (CA); Keith Humphries, Vancouver (CA); Jana Krosl, Montreal (CA)

Correspondence Address: BERESKIN AND PARR, 40 KING STREET WEST, BOX 401, TORONTO, ON, M5H 3Y2, CA

(73) Assignee: INSTITUTE DE RECHERCHES CLINIQUES DE MONTREAL

(21) Appl. No.: 10/530,413

(22) PCT Filed: Oct. 6, 2003
(86) PCT No.: PCT/CA03/01539

§ 371 Date: Jan. 9, 2006

(30) Foreign Application Priority Data
Oct. 8, 2002 (US) 60416545

(51) Int. Cl. C07H 21/04; C12P 21/06; C07K 14/475; C12N 5/06; C12N 5/08; C12N 15/87

(52) U.S. Cl. 435/69.1; 536/23.2; 435/366; 530/350; 435/455; 435/354

(57) ABSTRACT

The present invention relates to a stem cell expansion factor comprising a blocker which reduces expression level of at least one gene normally limiting HOX-induced expansion of stem cells, whereby reducing expression level of said gene enhances expansion of stem cells containing a HOX peptide. The present invention also relates to a composition for enhancing expansion of stem cells comprising an amino acid sequence having the activity of a HOX peptide, wherein said peptide being able to cross a cell membrane, and a blocker which reduces expression level of at least one gene normally limiting HOX-induced expansion of stem cells, whereby reducing expression level of said gene in the presence of a HOX peptide enhances expansion of stem cells. The preferred HOX peptide is HOXB4 and the preferred gene is PBX.

### BACKGROUND OF THE INVENTION

(a) Field of the Invention

The present invention relates to a stem cell expansion factor, a construct, a composition and a method thereof for enhancing stem cell expansion using a blocker which reduces expression level of at least one gene normally limiting HOX-induced expansion of stem cells.

(b) Description of Prior Art

In human, successful reconstitution of the blood system by hematopoietic stem cells (HSCS) depends of both the number and expansion of the transplanted HSCs. Cord blood is a rich source of HSCs but the number of cells available per each specimen is, in most cases, too low to allow a successful reconstitution of the blood system in adult humans. Therefore there is much interest to identify factors that would contribute to the expansion of HSCs.

Although considerable progress has been made towards identifying cytokine combinations that maintain or moderately expand HSCs in vitro (Conneally, E., et al., Proc. Natl. Acad. Sci. U.S.A. 94: 9836-9841, 1997; Miller, C. L. and Eaves, C. J., Proc. Natl. Acad. Sci. U.S.A. 94: 13648-13653, 1997), the culture conditions that allow for significant ex-vivo expansion of HSCs remain to be defined. A number of studies suggest that HSC expansion can be achieved by activation of cell intrinsic pathways. Activation of Notch-1 in response to soluble Jagged-1 added to culture media resulted in expansion of multipotent colony-forming cells (CFC) and maintenance of HSCs with lympho-myeloid repopulation potential (Varnum-Finney, B., et al., Nat. Med. 6: 1278-1281, 2000; Karanu, F. N., et al., J. Exp. Med. 192: 1365-1372, 2000). Increased self-renewal of human HSCs' was also documented in cultures supplemented with soluble Sonic Hedgehog (Shh) (Bhardwaj, G., et al., Nat. Immunol. 2: 172-180, 2001). However, the magnitude of these expansions still remains relatively modest.

The Hox genes are implicated in the regulation of hematopoiesis (Reviewed in Magli, M. C., et al., J. Cell Physiol. 173: 168-177, 1997), and contribute to the development of human leukemias (Reviewed in Buske, C. and Humphries, R. K., Int. J. Hematol. 71: 301-308, 2000).

A growing body of evidence suggests that HOXB4 displays interesting properties in hematopoiesis. In a mouse bone marrow transplantation model, retroviral overexpression of HOXB4 induced up to a 1000-fold net increase in the number of transduced HSCs (Sauvageau, G., et al., Genes Dev. 9: 1753-1765, 1995; Thorsteinsdottir, U., et al., Blood. 94: 2605-2612, 1999; Antonchuk, J., et al., Exp. Hematol. 29: 1125-1134, 2001), and promoted a significant increase in the rate and the magnitude of hematopoietic reconstitution when compared to cells transduced with a control retroviral vector (Antonchuk, J., et al., supra). Using immuno-compromised NOD-SCID mice, it was recently shown that HOXB4 produced similar effect on primitive human cells (Buske, C., et al., Blood. 100: 862-868, 2002). Recent studies have also demonstrated that HOXB4 induced a rapid 40-fold ex vivo expansion of mouse HSCs (Antonchuk, J., et al., Cell. 109: 39-45, 2002). The enhanced expansion of HOXB4-transduced HSCs occurs without impairment in the normal production of mature cells. Moreover, leukemic transformation is not seen in long-term recipients of HOXB4-transduced cells.

The DNA-binding capacity of HOXB4 is required for its HSC expanding capacity. However HOXB4 point mutant protein lacking the capacity to form cooperative DNA-binding with PBX protein also retains the capacity to induce similar expansion of HSCs as wild-type.

The expression levels of endogenous PBX1 proteins in HOXB4-overexpressing cells can be knocked down (reduced) with appropriate technique. These HSCs cells are referred to as PBX1^{K.D} HOXB4-expressing HSCs. They are at least 20-fold more competitive than those engineered to overexpress HOXB4 without undergoing this treatment. The PBX1^{K.D} HOXB4-expressing HSCs show normal in vivo differentiation and do not override the endogenous mechanisms involved in regulating the maximal number of HSCs present in a mouse and do not cause leukemia or myeloproliferation.

It would therefore be highly desirable to be provided with a stem cell expansion factor, a construct, a composition and a method thereof for enhancing stem cell expansion using a blocker which reduces expression level of at least one gene normally limiting HOX-induced expansion of stem cells.

### SUMMARY OF THE INVENTION

One aim of the present invention is to provide a stem cell expansion factor, a construct, a composition and a method thereof for enhancing stem cell expansion using a blocker which reduces expression level of at least one gene normally limiting HOX-induced expansion of stem cells.

In accordance with one embodiment the present invention, there is provided a stem cell expansion factor comprising a blocker which reduces expression level of at least one gene normally limiting HOX-induced expansion of stem cells, whereby reducing expression level of said gene enhances expansion of stem cells containing a HOX peptide.

In accordance with one embodiment the present invention, there is provided a nucleic acid construct for enhancing stem cells expansion, the construct comprising a first nucleic acid sequence for expression of a HOX peptide, wherein the peptide being able to cross a cell membrane, and a second nucleic acid sequence blocking expression of at least one gene normally limiting HOX-induced expansion of stem cells, whereby reducing expression level of the gene in the presence of a HOX peptide enhances expansion of stem cells.

In accordance with one embodiment the present invention, there is provided a composition for enhancing expansion of stem cells comprising an amino acid sequence having the activity of a HOX peptide, wherein the peptide being able to cross a cell membrane, and a blocker which reduces expression level of at least one gene normally limiting HOX-induced expansion of stem cells, whereby reducing expression level of the gene in the presence of a HOX peptide enhances expansion of stem cells.

In accordance with one embodiment the present invention, there is provided a composition for enhancing expansion of stem cells comprising a nucleic acid sequence for over-expression of a HOX peptide, and a blocker which reduces expression level of at least one gene normally limiting HOX-induced expansion of stem cells, whereby reducing expression level of said gene in the presence of a over-expressed HOX peptide enhances expansion of stem cells.

In accordance with one embodiment the present invention, there is provided method for enhancing expansion of stem cells, which comprises treating stem cells with an effective amount of a factor of the present invention, or an effective amount of a composition of the present invention for a time sufficient to allow expansion of said stem cells.

In accordance with one embodiment the present invention, there is provided the use of a factor of the present invention, a construct of the present invention, or a composition of the present invention for the preparation of a medicament for restoring hematopoietic capability of a patient.

The blocker may be selected from the group consisting of an antisense, an antibody, a SiRNA, a peptide and a chemical compound.

The gene may be a PBX gene and the blocker may be a nucleic acid sequence blocking PBX expression. The preferred PBX gene is PBX1.

The blocker may be an antisense DNA to PBX1.

The preferred stem cells are hematopoietic stem cells, more preferably human or mouse hematopoietic stem cells.

In another embodiment of the present invention, the HOX peptide is a HOXB4 peptide.

In one embodiment of the invention, the amino acid sequence comprises an HIV-derived peptide able to cross a cell membrane. Such HIV-derived peptide may consist for example of a NH2-terminal protein transduction domain (PTD) from a transactivating protein.

In accordance with the present invention, there is further provided a method for enhancing expansion of stem cells, the method comprising delivering to stem cells an effective amount of a factor as defined above or an effective amount of a composition containing the factor, wherein HOXB4 is present in said stem cells and wherein PBX expression is blocked in said stem cells. The method may further comprise a step of delivering an amino acid sequence having the activity of a HOX peptide encoded by a HOX nucleotide sequence.

One aim of the present invention is to provide material and methods to generate hematopoietic stem cells (HSC) having high expanding capacity.

In accordance with a preferred embodiment of the present invention there is provided a procedure to generate HSCs that over-express the gene HOXB4 whereas the level of PBX is suppressed. This particularly engineered type of HSC is many times more competitive than a previously reported line of HSC whereas HOXB4 is over-expressed but the level of PBX is not manipulated.

All publications, patents and patent applications are herein incorporated by reference in their entirety to the same extent as if each individual publication, patent or patent application was specifically and individually indicated to be incorporated by reference in its entirety.

### BRIEF DESCRIPTION OF THE DRAWINGS

FIG. 1 illustrates Pbx gene expression in primitive subpopulations of mouse bone marrow cells.

FIG. 2 illustrates some retroviral vectors suitable for suppressing PBX1.

FIG. 3 illustrates the results of flow cytometry analyses and Southern blot analyses of bone marrow cells.

FIG. 4 illustrates Western blot analyses of HSC cultures.

FIG. 5 illustrates Western blot analyses of PBX1 protein levels in samples taken from mice transplanted with different types of HSCs.

FIG. 6 illustrates the expansion of transduced primary bone marrow cells in liquid culture initiated with different HSCs.

FIG. 7 illustrates in vitro expansion of different HSCs.

FIG. 8 illustrates the competitive reconstitution with HOXB4-transduced bone marrow cells.

FIG. 9 illustrates the competitive reconstitution with HOXB4 and HOXB4 plus a/s PBX1-transduced cells.

FIGS. 10A to 10C illustrate flow cytometric analysis of hematopoietic cells from bone marrow (FIG. 10A), spleen (FIG. 10B) and thymus (FIG. 10C) of mice transplanted with different HSCs.

FIGS. 11A to 11D illustrate the numbers of myeloid (FIGS. 11A and 11B) and lymphoid (FIGS. 11C and 11D) CFC content of bone marrow (FIGS. 11A and 11C) and spleen (FIGS. 11B and 11D) in mice transplanted with different HSCs.

FIG. 12 illustrates the distribution of bone marrow and spleen derived myeloid CFC in mice.

FIG. 13 illustrates Southern blot analysis of proviral integrations in DNA isolated from myeloid clones derived from HSC recipients

FIG. 14 illustrates the contributions of HOXB4(GFP⁺) and a/s PBX1+HOXB4(GFP+/YFP⁺)-transduced HSC to reconstitution of hematopoietic system of secondary recipients.

FIG. 15 illustrates Southern blot analysis of proviral integrations in DNA isolated from bone marrow, spleen and thymus of a mouse transplanted with 10% HOXB4, 13% a/s PBX1+HOXB4, and 24% a/s PBX1-transduced bone marrow and its secondary recipients sacrificed 16 weeks post transplantation.

FIG. 16 illustrates Southern blot analysis of myeloid clones isolated from secondary recipients of HOXB4 and a/s Pbx+HOXB4-transduced cells.

FIG. 17 illustrates the comparison of CRU recovery following competitive reconstitution in groups receiving different HSCs.

### DETAILED DESCRIPTION OF THE PREFERRED EMBODIMENT

Following is a detailed description of the procedure to generate HSCs in which the expression of PBX1 is suppressed while HOXB4 is over-expressed resulting in greatly enhanced expansion of the HSC in vitro as well as in vivo. The scientific reasoning and the logic for the attempted procedures are also given for clarity. The present invention has been illustrated with respect to expression of PBX1. Of course, one skilled in the art will understand that any gene of the PBX family could also have been used. In fact, there is a strong homology between the various member of the PBX family. Pbx1 is a gene that encodes for a protein which is known to form cooperative DNA binding with HOX proteins. There are at least 4 Pbx genes (Pbx1, Pbx2, Pbx3 and Pbx4) in the genome of mammals including human and mice. All 4 Pbx genes encode for proteins with high sequence similarity over the entire region of the proteins. Since PBX proteins are so similar, it is expected that they will perform similar functions. Therefore, and depending on the cell type analyzed, it is expected that any member of the PBX family has the potential to interfere with the stem cell expanding function of HOXB4. All PBX genes are therefore included in the present invention.

HOXB4 can bind DNA either as a monomer or cooperatively as a heterodimer with PBX proteins. A HOXB4 point mutant protein lacking the capacity to form cooperative DNA-binding interaction with PBX1 can still lead to HSC expansion. This suggests that endogenous PBX is not required for the HOXB4-induced expansion of HSCs.

One essential part of the invention is the process to knock down the levels of PBX1 in primary bone marrow cells.

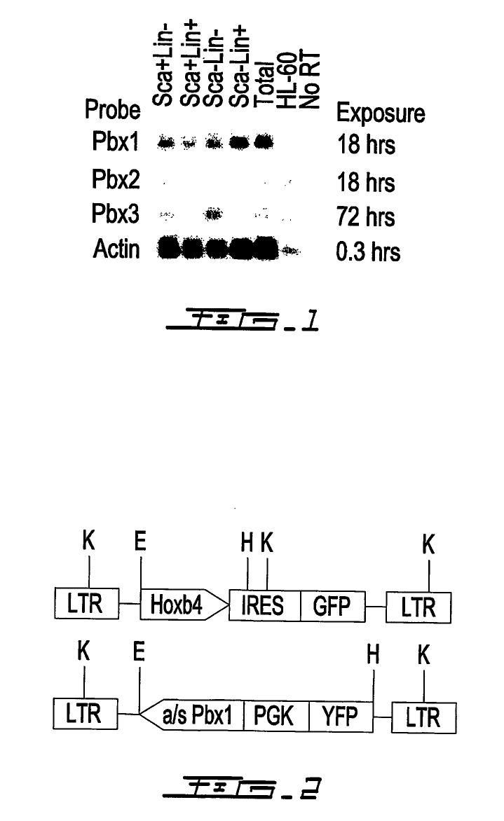

Experiments were performed to determine whether lowering levels of PBX genes expressed in HSCs might further enhance the HOXB4-induced expansion of these cells. Semi-quantitative RT-PCR was used to measure the relative mRNA levels of all 3 known PBX genes expressed in phenotypically pure subpopulation of bone marrow cells highly enriched for HSC activity. Although expression of all 3 PBX genes was detected in the most primitive fraction (Sca1⁺Lin⁻), expression of PBX1 appeared to be predominant. In FIG. 1 total RNA isolated from the indicated subpopulations of bone marrow was subjected to reverse transcription and global amplification. Probes used for Southern blot analyses of the amplified cDNAs are indicated to the left, and exposure times of autoradiograms are stated in the right of figure. Probes specific for each member of PBX family were box-less fragments of the corresponding CDS which in Southern blot analysis identified genomic PBX-comprising bands of the predicted sizes. Note that Pbx 3 blot required 3-times longer exposure than Pbx1 and Pbx 2 blots.

Previous studies demonstrated that retroviral transduction of antisense cDNA to PBX1 represented a very effective tool for suppressing endogenous levels of this protein (Krosl, J. and Sauvageau, G., Oncogene. 19: 5134-5141, 2000). Mouse bone marrow cells were infected with recombinant retroviruses encoding antisense PBX1 cDNA and yellow fluorescent protein (a/s PBX1-YFP), or HOXB4 and green fluorescent protein (HOXB4-GFP), or with a combination of both vectors See FIG. 2 for description of vectors.

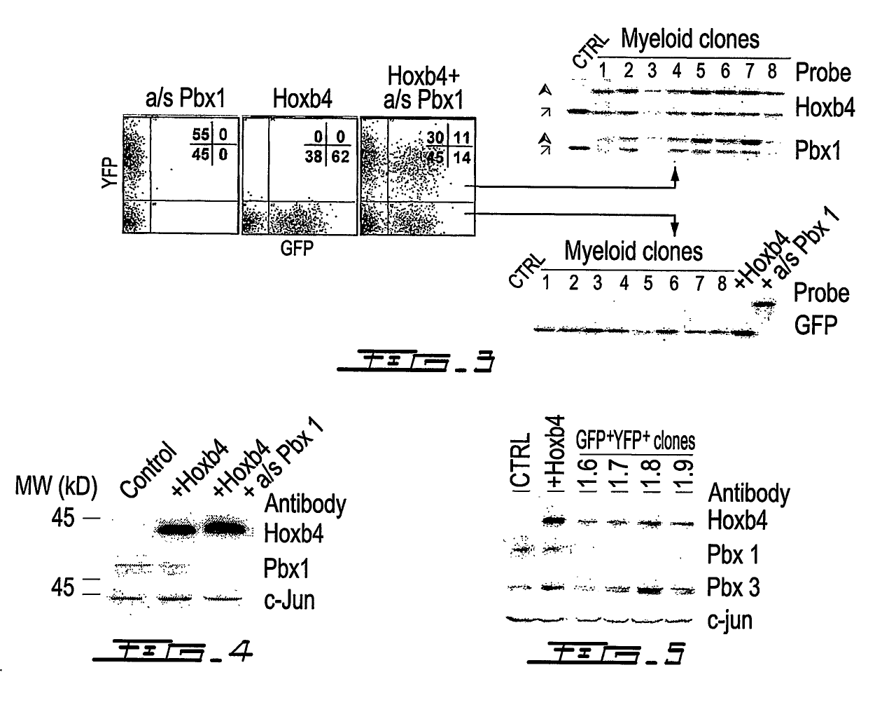

Representative examples of gene transfer efficiencies, assessed by flow cytometric analyses of YFP and GFP expression, are shown in FIG. 3. In this figure the left panel presents the results of flow cytometric analyses of YFP, GFP and YFP+GFP expression in populations of bone marrow cells derived from 5-FU-trated mice. Analyses were done one day after the bone marrow cells were harvested from co-cultures with a/s PBX1(YFP⁺), HOXB4(GFP⁺), and a/s PBX1 plus HOXB4 (GFP⁺ and YFP⁺) retrovirus producing cells. It is seen that in populations of cells co-infected with a/s PBX1-YFP and HOXB4-GFP, the efficiencies of double infection (YFP⁺/GFP⁺) were 9-14%, and singly infected cells (i.e., YFP⁺ or GFP⁺) represented 35-44% and 12-15%, respectively, of total cell populations.

The Right panel of FIG. 3 shows Southern blot analyses of proviral integrations in DNA isolated from clonal populations initiated with GFP⁺/YFP⁺ (right top panel) or GFP⁺ (right lower panel) cells. One day after recovery from co-culture with cells producing recombinant HOXB4-GFP and a/s PBX1-YFP retroviruses, cells from GFP⁺/YFP⁺ and GFP⁺ windows were sorted using MoFlo and plated in standard methylcellulose. Multilineage mixed colonies (CFU-GEMM) were plucked randomly on day 7, and expanded in liquid culture to yield total of 0.7-1×10⁷ cells. DNA isolated from these clones was digested with Kpn I, which releases the 4.4 kb a/s PBX1-YFP proviral genome, and cuts in LTRs and IRES of HOXB4-GFP provirus to generate the 2.3 kb HOXB4- and the 1.7 kb GFP-comprising fragments. Probes used are indicated to the right. The results confirmed that all GFP⁺/YFP⁺ clones originated from [HOXB4-GFP+a/s PBX1-YFP]-transduced progenitors (FIG. 3, right upper panel), and that GFP⁺ cells comprised only HOXB4-GFP-transduced cells (FIG. 3, right lower panel).

Western blot analyses as presented in FIG. 4 showed that both, GFP⁺ and GFP⁺/YFP⁺ populations expressed high levels of HOXB4 protein (FIG. 4, top panel), and that the antisense approach was effective in knocking down levels of PBX1 protein in the of GFP⁺/YFP⁺ BM cells (FIG. 4, third lane of middle panel).

For simplicity, cells expressing lower levels of PBX1 as a result of the antisense retroviral vector will be identified as “PBX1^{K.D.}” (for Knock Down PBX) and those engineered to express HOXB4 and antisense PBX1 as “HOXB4-PBX1^{K.D.}” cells.

FIG. 5 presents Western blot analysis of HOXB4 and PBX1 protein levels in myeloid clones isolated Group II recipients. CTRL, clone negative for HOXB4 and PBX1 proviral integrations; +HOXB4, clone harboring HOXB4 provirus (see FIG. 13, right panel, Clone #2.1); Clone numbers indicated at the top correspond to the clones genotyped in FIG. 13, left panel. The left panel of FIG. 5 shows clone negative for HOXB4-GFP and a/s PBX1-YFP proviral integrations; +Hoxb4, clone harboring HOXB4-GFP provirus. The right panel shows myeloid clones generated by HOXB4-GFP and a/s PBX1-YFP-transduced CFC. This clearly demonstrates the low level of PBX protein achieve with the antisens approach of the present invention.

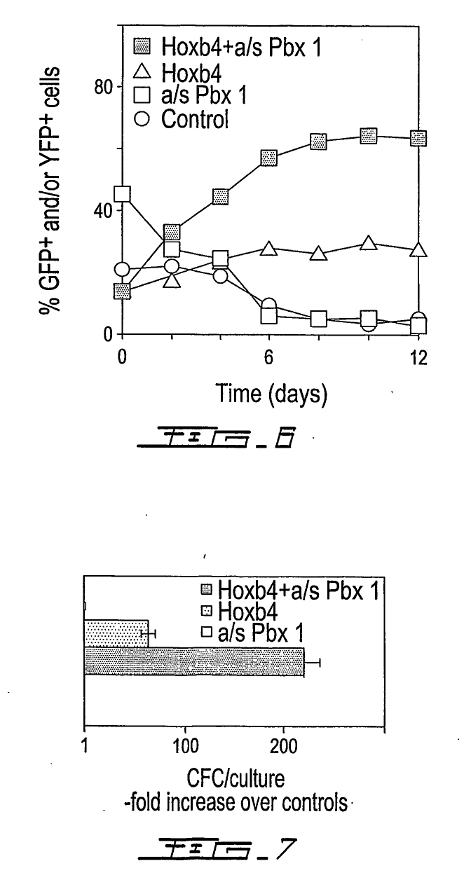

There are proofs that HOXB4-PBX1^{K.D.} bone marrow cells have higher in vitro proliferation potential than those overexpressing HOXB4. HOXB4 overexpression confers a significant in vitro proliferation advantage to transduced bone marrow cells (Antonchuk, J., et al., Exp. Hematol. 29: 1125-1134, 2001). Using an in vitro competitive proliferation assay initiated with 4 different populations of transduced-BM cells (see FIG. 6), HOXB4-PBX1^{K.D.} BM cells expanded much better than HOXB4 cells: within 3 days, the proportions of HOXB4-PBX1^{K.D.} cells (black squares) increased by approximately 2-fold above the values determined for HOXB4 cells and became by day 9 the predominant cell type representing over 60% of the population. In contrast, the proportion of PBX1^{K.D.} (YFP⁺) cells decreased steadily with time in culture and were barely detectable by day 12 clearly indicating that PBX1^{K.D.} cells had no proliferative advantage over non-transduced cells.

The in vitro expansion of colony-forming cells (CFC) derived for each of the 4 populations (i.e., HOXB4-PBX1^{K.D.}; HOXB4; PBX1^{K.D.}; untransduced) was assessed in liquid cultures initiated with equal numbers of sorted GFP⁺, GFP⁺/YFP⁺, YFP⁺ and GFP⁻/YFP⁻ cells. By day 12 of the culture, HOXB4-PBX1^{K.D.} CFC increase ˜2.5-fold above the levels determined for HOXB4 cells (FIG. 7), for a net 220-fold expansion over their starting numbers. Expansion of PBX1^{K.D.} CFC was similar to that seen with untransduced cells (i.e., 6-7-fold).

Importantly, the competitiveness of HOXB4-PBX1^{K.D.} over HOXB4 overexpressing cells was not due to a negative feedback mechanism since the latter expanded similarly whether they were grown in the presence or absence of HOXB4-PBX1^{K.D.} cells.

The following paragraphs furnish evidence that PBX1^{K.D.} increases the repopulating potential of bone marrow cells overexpressing HOXB4.

Two different groups of BM transplantation chimeras were generated by transplanting a mixture of HOXB4 cells together with untransduced cells (group I), or a mixtures of cells comprising the same 4 populations detailed in the previous section (group II). For both groups, the relative contribution of each subpopulation to the bone marrow graft is indicated in FIG. 8 (t=0: representative group of 4 mice) and summarized for all 3 experiments in histograms at the bottom of FIG. 8 (see t=0). In group I recipients (inoculum comprised ˜10% HOXB4-GFP-transduced cells), GFP⁺ cells represented 25±4% of PBL at 7 weeks post reconstitution as shown by FASC profiles of four recipients (FIG. 8, center), and increased to 42±6% by week 30 (FIG. 8, bottom) clearly demonstrating the repopulating advantage of these cells. Control GFP-transduced cells in similar conditions reconstituted less than 3% of the hematopoietic system at these time points.

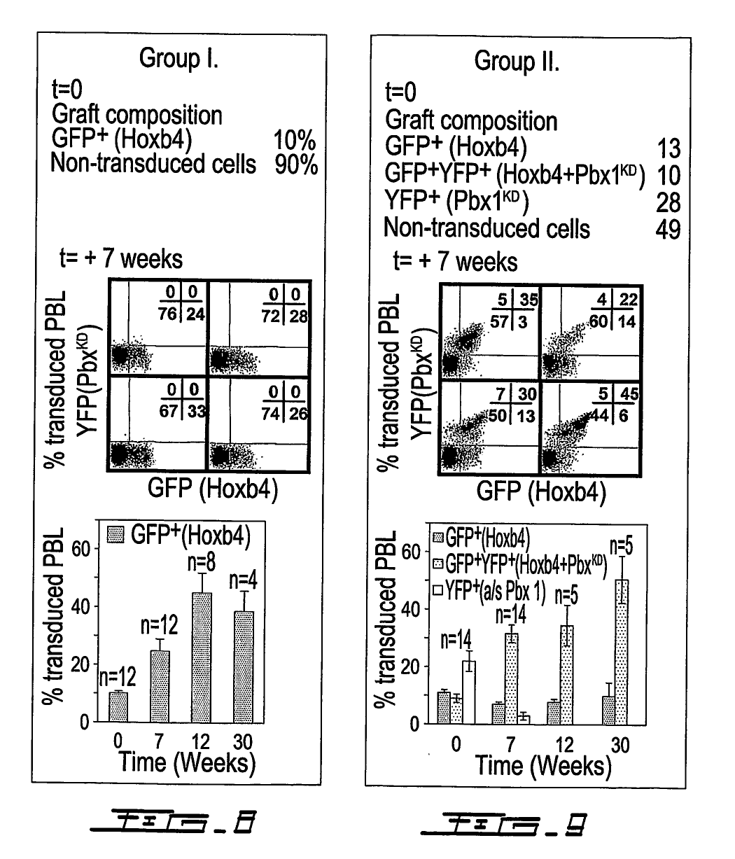

Description of the composition of a representative bone marrow graft transplanted in primary Group II recipients received a mixture of (1) HOXB4-PBX1^{K.D.}; (2) HOXB4-overexpressing; (3) PBX1^{K.D.} and (4) untransduced cells (FIG. 9, top). A summary of the graft composition for 14 different primary recipients (from 3 different experiments) is shown at the bottom of right panel in FIG. 8 (t=0). In all cases HOXB4-transduced cells and HOXB4-PBX1^{K.D.} cells each represented ˜10% of the inoculum and PBX1^{K.D.} cells ˜25%. The proportions of YFP⁺/GFP⁺ cells (representing the HOXB4-PBX1^{K.D.} cells) increased from ˜10% to 32±6% by week 7 (FIG. 9, center), and to 51±7% by week 30, while proportions of GFP⁺ cells (HOXB4 cells) never increased above the starting 10% (black bars in FIG. 9, bottom). Although YFP⁺ cells (HOXB4-PBX1^{K.D.} cells) represented up to 28% of the initial graft, their numbers decreased to less than 7% at early time points and became undetectable beyond 12 weeks after transplantation (white bars in FIG. 9, bottom). YFP⁻/GFP⁻ (i.e., untransduced) cells seemed to account for up to 50% of the reconstitution. However, as shown bellow vast majority of CFCs originated from HOXB4-PBX1^{K.D.} or HOXB4 cells. This observations suggested that either expression levels of marker genes in the apparently GFP⁻YFP⁻ PBL remained bellow the threshold of FACS detection, or that significant proportion of the integrated proviruses was inactivated.

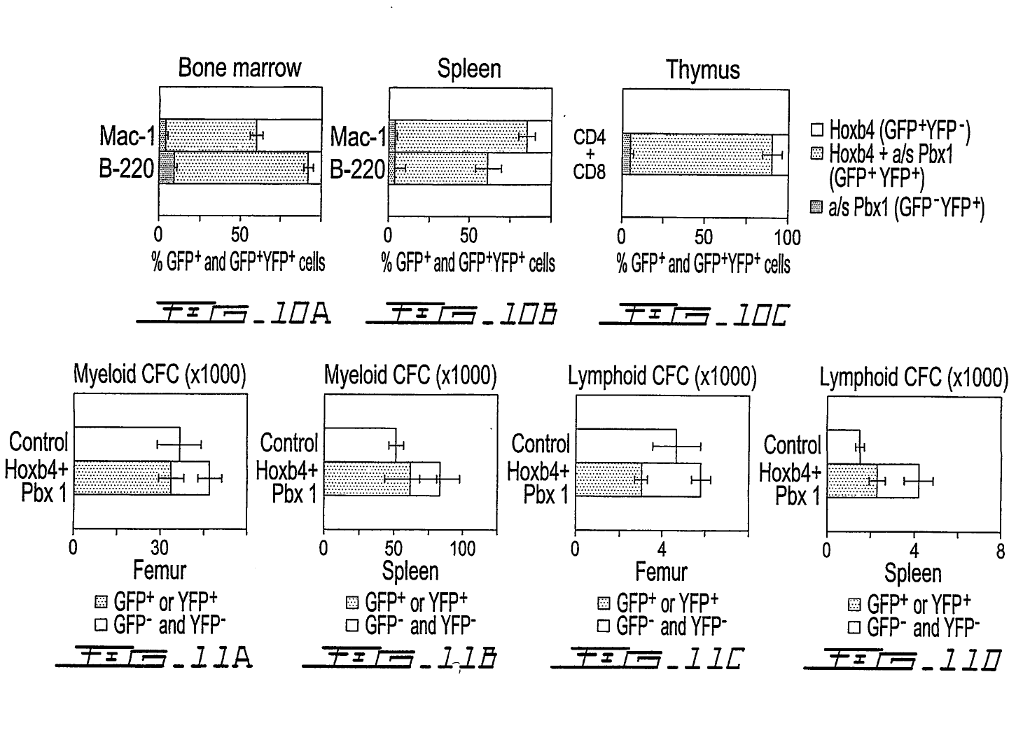

All group I and II primary recipients remained healthy for up to 9 months of observation, and had normal numbers and distribution of myeloid and lymphoid cells in their bone marrow, spleen and thymus. The proportion of cells expressing GFP and/or YFP in each of these organs is shown in FIG. 10 for 4 mice (>16 weeks post transplantation). GFP+YFP+(HOXB4-PBX1^{K.D}) cells which represented about 1/10^{th} of the bone marrow graft accounted for 60-75% of bone marrow and spleen derived Mac-1+cells; ˜45-75% of bone marrow and spleen-derived B cells and ˜80-85% of CD4⁺/CD8⁺ thymocytes (see gray bars in FIG. 10). Consistent with the low proportion of GFP⁺YFP⁻ (HOXB4-transduced) cells in the peripheral blood from these mice, only a minor contribution (5-7%) of these cells could be detected in the bone marrow, spleen and thymus from these primary recipients (FIG. 10, black bars).

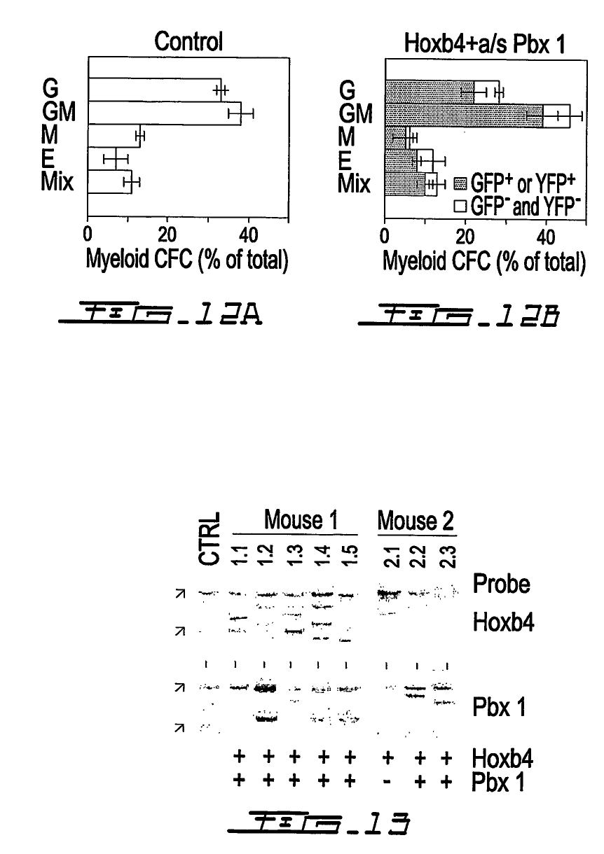

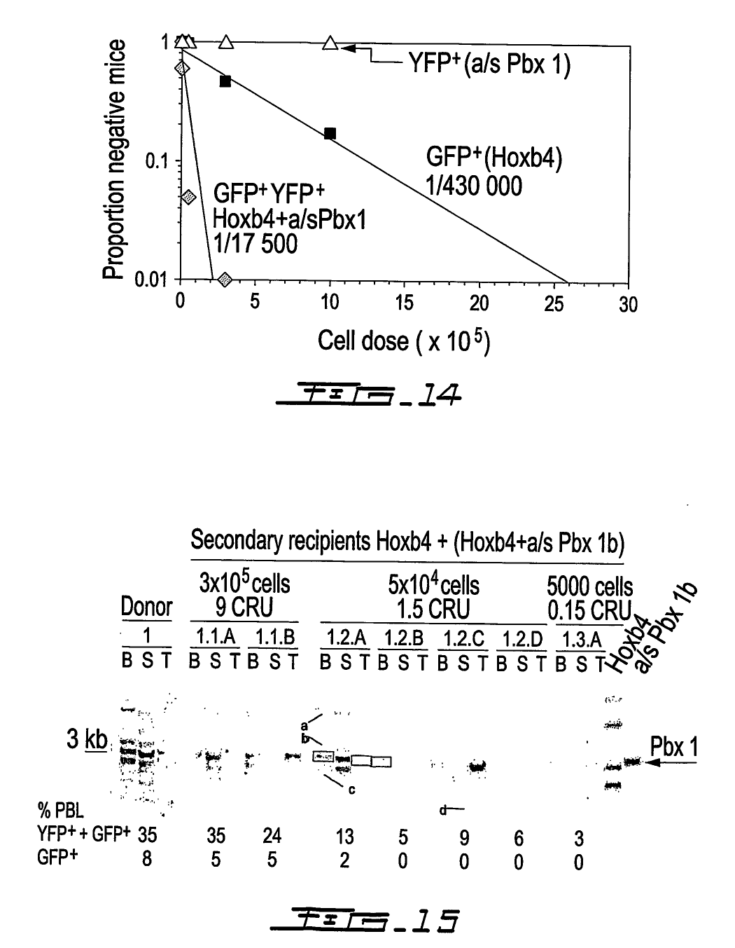

As often seen in recipients of HOXB4-transduced cells (genes dev., 1995), these complex hematopoietic chimeras had a slight elevation (not statistically different with the number of mice analyzed: n=6) in the numbers of spleen myeloid and B-lymphoid colony-forming cells (CFC) (FIG. 11). The distribution of the various types of CFC was not different between GFP⁺ or YFP⁺ and control cells (FIG. 12). Since it was impossible to distinguish by epifluorescence between GFP and/or YFP-expressing cells, we analyzed DNA isolated from individual randomly plucked colonies for the presence of the integrated HOXB4 and a/s PBX1 provirus (FIG. 13, representative clones). In total, 44/53 or 80% of myeloid CFCs derived from 3 recipients originated from doubly-transduced cells (HOXB4-PBX1^{K.D}), and only 4/53 or 7% from HOXB4-transduced cells. Surprisingly, only 5/53 or 9% of randomly generated clones harbored neither HOX4 nor a/s PBX1 provirus, suggesting a minimal contribution of non-transduced transplant-derived and endogenous host cells to reconstitution of hematopoietic system.

Together, these results indicated that: (1) HOXB4-PBX1^{K.D.} cells were in vivo much more competitive than HOXB4-transduced cells; (2) HOXB4-transduced cells were more competitive than non-transduced or PBX1^{K.D} cells. Repopulation advantage conferred by PBX1^{K.D} thus occurred only in the context of HOXB4 overexpression. These studies, however, did not clarify whether the in vivo competitive advantage of HOXB4-PBX1^{K.D} cells occurred at the level of stem cells, or in more mature progenitors.

The expansion of HOXB4-PBX1^{K.D} HSCs was also studied in vivo. The in vivo expansion was assessed using the CRU Competitive Repopulation Unit) assay (Szilvassy, S. J., et al., Proc. Natl. Acad. Sci. U.S.A. 87: 8736-8740, 1990; Sauvageau, G., et al., Genes Dev. 9: 1753-1765, 1995; Thorsteinsdottir, U., et al., Blood. 94: 2605-2612, 1999) and compared to the expansion of HOXB4-transduced cells within the same recipient. At 16 weeks post transplantation, bone marrow cells were harvested from the hematopoietic chimeras (group II) and transplanted at decreasing dilutions into secondary recipients. Contribution of the transduced (either GFP⁺, GFP⁺/YFP⁺ or YFP⁺) cells to lymphoid and myeloid reconstitution of secondary recipients was determined by flow cytometry at 16 weeks post transplantation and, estimation of CRU numbers was performed using the maximum likelihood method as described (Szilvassy, S. J., et al., Proc. Natl. Acad. Sci. U.S.A. 87: 8736-8740, 1990). By that time, the frequency of HOXB4-PBX1^{K.D} CRU was 1/17,500 (FIG. 14) representing approximately 10⁴ CRU per mouse, considering that the hematopoietic system of an adult mouse contains ˜2×10⁸ cells. Since our primary recipients received approximately 10 HOXB4-PBX1^{K.D} CRUs (Table 1).

TABLE 1
CRU recovery at 16 weeks post transplantation

| | | Group I. | Group II. |
|---|---|---|---|
| No. of | | 92 | 106 |
| transplanted | | (61-152) | (70-175) |
| CRU¹(95% Cl) | | | |
| Composition of | GFP⁺ | 12 | 14 |
| transplant² | GFP⁺ + YFP⁺ | — | 11 |
| | YFP⁺ | — | 30 |
| | GFP⁻ + YFP− | 80 | 51 |
| CRU frequency | GFP⁺ | 1/26,400 | 1/430,000 |
| (95% Cl)³ | | (1/19,600-1/35,800) | (1/328,600-1/566,400) |
| | GFP⁺ + YFP⁺ | — | 1/17,500 |
| | | | (1/13,200-1/23,100) |
| | YFP⁺ | — | >1/1,000,000 |
| | GFP⁻ + YFP⁻ | ND | ND |
| CRU/mouse⁴ | GFP⁺ | 7,576 | 465 |
| | | (5,586-10,204) | (353-609) |
| | GFP⁺ + YFP⁺ | | 11,428 |
| | | | (8,658-15,151) |
| | YFP⁺ | — | >200 |
| | GFP⁻ + YFP⁻ | — | ND |
| | | ND | |
| % of normal | GFP⁺ | 76 | 4.6 |
| | GFP⁺ + YFP⁺ | — | 110 |
| | YFP⁺ | — | >2 |
| | GFP⁻ + YFP⁻ | ND | ND |

CRU frequency in 5-FU derived bone marrow cells after 2 day prestimulation was 1/2670 (1/1680 to 1/4218, 95% Cl). Numbers of transplanted CRU represent progeny of cells introduced into co-culture with retroviral producers.
Proportions of transduced CRU were calculated from infection efficiencies determined by flow cytometric analysis of GFP, YFP and GFP + YFP expression by bone marrow cells 1 day after recovery from co-culture with retroviral producers
CRU frequencies represent mean values determined 2 (group I) and 3(group II) recipients using limiting dilution analysis
Values shown were calculated from CRU frequencies, and assumption that one femur represents ˜10% of total bone marrow.

These data suggest an in vivo net expansion of up to 1000-fold for HOXB4-PBX1^{K.D} CRUs. CRU frequency of HOXB4-transduced cells from these same primary recipients was estimated at only 1/430 000, representing at best ˜500 CRU in these mice for an estimated expansion in the range of 50-fold. CRU frequency of PBX1^{K.D} cells could not be determined because none of the numerous secondary recipients (n=90) were reconstituted with a CRU derived from PBX1^{K.D} populations, although they represented ˜25% of the cells transplanted per primary recipient. For group II recipients, CRU frequencies were also determined at 32 weeks post-transplantation and found within similar numbers to those shown at 16 weeks indicating that the expansion of HOXB4-PBX1^{K.D} CRUs has stabilized over time. Together, results of these CRU assays indicate that the major in vivo competitive advantage of HOXB4-PBX1^{K.D} over HOXB4 cells occurred at the level of HSCs.

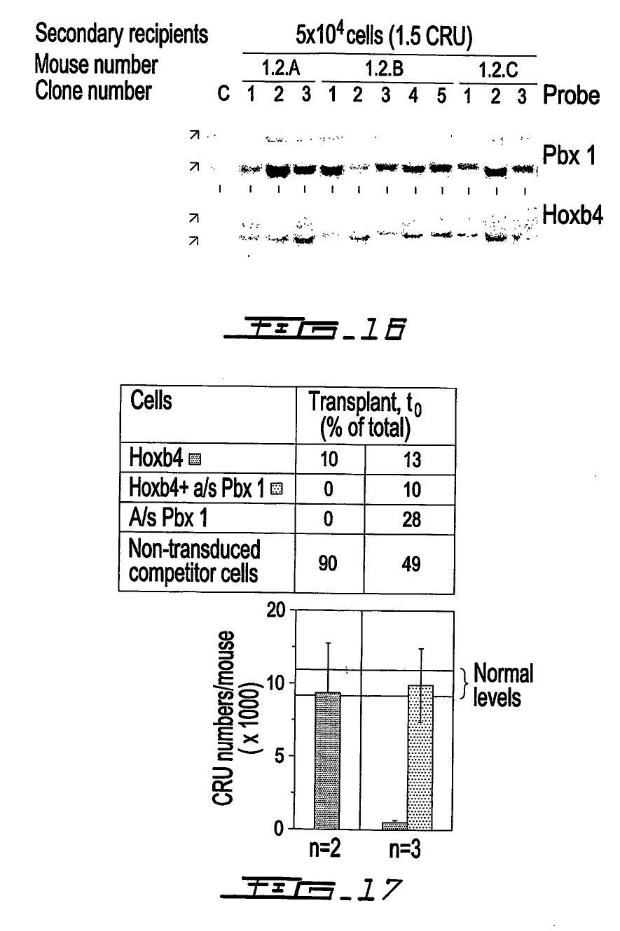

To verify that regeneration of HOXB4-PBX1^{K.D} CRUs in our hematopoietic chimeras was indeed mediated by pluripotent doubly transduced HSCs, and to determine the number of cellular clones that contributed to reconstitution, Southern blot analyses of proviral integrations in DNA isolated from bone marrow, spleen and thymus of primary recipients, and the corresponding secondary recipients used for CRU assays were performed (FIG. 15, a representative Southern blot analysis). To facilitate distinction between the two integrated proviruses (i.e., HOXB4-GFP and a/s PBX1-YFP), DNA was digested with EcoR I and Hind III, which cut HOXB4-GFP provirus upstream of GFP, such that each proviral integration generated a distinct autoradiographic band (clonal analysis), and also release from a/s PBX1 provirus a 3 kb PBX1-YFP cassette (see FIG. 2 for a diagrammatic representation of the 2 retroviruses and see FIG. 15 for the common signal at 3 Kb).

In the bone marrow, spleen and thymus of primary recipient and secondary recipients transplanted with 5×10³-1×10⁶ cells, multiple HOXB4 proviral integrations could be detected. Variations in autoradiographic intensities of these bands suggest that multiple cellular clones (at least 4, see “a, b, c and d” in FIG. 15), rather than few clones with several proviral integrations, contributed to reconstitution of these tissues (FIG. 15, left and central panel). The pluripotent nature of reconstituting HSC becomes apparent in mice transplanted with ˜1 CRU (FIG. 15, right panel), where the same proviral integration pattern can be detected in bone marrow and thymus of secondary recipients 1.2.A, 1.2.B, 1.2.C and 1.2.D. In addition, self-renewal division in primary recipients of several of these clones was documented, since one cellular clone repopulated more than one secondary recipient (see for example clone a or c in several of the mice). To complement this study, DNA was also isolated from clonogenic progenitors derived from the bone marrow of these mice. A total of 36 myeloid clones were analyzed from the various secondary mice presented in FIG. 15, and Southern blot analysis revealed that all (36/36) originated from doubly transduced HSCs (FIG. 16, representative analysis). Similar repopulation outcomes were determined for all 3 primary recipients that were analyzed.

The proliferative potential of individual CRU can be estimated in mice transplanted at limiting dilution, where the activity of single CRU contributes to the production of 1-5% of mature myeloid and lymphoid cells. Flow cytometric analyses of secondary recipients from group II primary mice revealed that at limited dilution HOXB4-PBX1^{K.D} CRUs generated on average 4±2% GFP⁺/YFP⁺ PBL (mean value±SD, n=30). The competitive nature of HOXB4-PBX1^{K.D} HSCs was thus not due to an increased output of individual stem cells, but rather to a preferential and competitive expansion of the stem cell population in these primary recipients.

The evidence for strict controls of the maximal HSCs pool size provides some understanding of the competitive nature of HOXB4-PBX1^{K.D} vs HOXB4-transduced HSCs. As shown in FIG. 14 and Table 1 CRU frequency of the various populations varied considerably in our mixed chimeras, from 1/17,500 cells for HOXB4-PBX1^{K.D} cells to 1/430,000 cells for HOXB4-transduced cells. The number of HOXB4-transduced CRU detected in HOXB4-PBX1^{K.D} vs HOXB4 competitive set-up (FIG. 17, right panel, black bar) was much lower than values determined for group I recipients reconstituted with ˜10% HOXB4-transduced cells and 90% untransduced cells (FIG. 17, right panel, black bar), or the values determined in our previous experiments (Antonchuk, J., et al., Exp. Hematol. 29: 1125-1134, 2001; Antonchuk, J., et al., Cell. 109: 3945, 2002; Sauvageau, G., et al., Genes Dev. 9: 1753-1765, 1995; and Thorsteinsdottir, U., et al., Blood. 94: 2605-2612, 1999). Frequencies of HOXB4-PBX1^{K.D} CRUs in group II mice, were, however, comparable to the values determined for the transduced CRUs in group I recipients (FIG. 17, compare black bar in left panel to gray bar in right panel), and to the previously reported values (Refs), and were at least 20-times higher than frequencies of HOXB4 cells within the same recipient (FIG. 17, right panel). In competition with cells possessing superior reconstitution potential (i.e. HOXB4-PBX1^{K.D} CRUs), the HOXB4-transduced CRUs thus limited their expansion to levels normally seen with untransduced cells, suggesting the existence of a mechanism that regulate the total size of the HSC compartment in mice.

Together, these studies demonstrate a new avenue in HOXB4-induced stem cell expansion and uncover a non-cell autonomous mechanism that limits the expansion of HOXB4-transduced cells in vivo.

HOXB4 has been characterized as a unique factor capable of inducing considerable in vivo and ex vivo expansions of HSCs. This disclosure demonstrates that the HOXB4-specific capacity to expand HSCs can be further increased by ˜20-fold using an antisense approach to PBX1. This effect was observed on numerous clones isolated from several mice. All recipients of HOXB4-PBX1^{K.D.} HSCs remained healthy and without any detectable sign of hematopoietic disease for up to 9 months of observation, demonstrating that the transduced HSCs which underwent a significant in vivo expansion remained functionally unimpaired. These characteristics, coupled with the ability of HOXB4 to promote ex vivo expansion of HSC (Antonchuk, J., et al., Cell. 109: 39-45, 2002) may have major implications on the development of new strategies based on stem cell transplantation. On a more fundamental basis, these studies revealed that expansion of HOXB4-overexpressing cells in vivo can be inhibited in the presence of a more competitive population such as HOXB4-PBX1^{K.D.} cells. In this competitive setting, HOXB4-transduced cells failed to exert their stem cell expanding capacity, and regenerated in recipients only a minor fraction of HSC pool. Interestingly, the highly competitive HOXB4-PBX1^{K.D.} HSC did not repopulate the HSC pool size to values greater than those observed with HOXB4-transduced cells, but did reach values that characterize normal, unmanipulated animals, indicating the presence of a non-cell autonomous mechanism that regulates of the number of HOXB4-transduced HSC in vivo.

The mechanism(s) that limit the in vivo expansion of HOXB4-PBX1^{K.D.}- and HOXB4-transduced cells remain to be identified. The proliferation rates of HOXB4-transduced cells cultured in the presence or absence of HOXB4-PBX1^{K.D.} cells were comparable, suggesting that this mechanism may not be active during the ex vivo expansion. Our study, is however, did not directly assess the in vitro expansion of HOXB4-transduced HSC cultured in the presence and absence of HOXB4-PBX1^{K.D.}-transduced HSC, such that at this point a possibility of some mechanism that specifically limits self renewal divisions of HOXB4-transduced HSC in vivo and in vitro can not be excluded. To develop methods for exploiting HOXB4 as a HSC-specific mitogen it will be important to determine whether the net 40-fold ex vivo expansion of HOXB4-transduced HSC we reported recently can be further enhanced by as much as 20-fold in the context of PBX1^{K.D.}. It will also be interesting to determine whether other approaches, such as PBX1 siRNA, could achieve effects comparable to antisense approach reported herein.

In agreement with the previously reported observations (DiMartino, J. F., et al., Blood. 98: 618-626, 2001), PBX1^{K.D} cells showed no proliferative advantage; it is only in the context of HOXB4 overexpression that the PBX1^{K.D.} gains major significance. Our observations thus suggest that proliferation associated with self-renewal of HSC can be modulated by a subset of HOXB4 responsive genes in a PBX1 independent manner, and indicate that PBX1 acts as a negative regulator of HOXB4-induced HSC proliferation. PBX1^{-/-} mice could represent an alternative model for studying the role of PBX1 in HOXB4-induced stem cell expansion. PBX1^{-/-} generates, however, embryonic lethal phenotype characterized by generalized hypoplasia associated with the absence of spleen (DiMartino, J. F., et al., Blood. 98: 618-626, 2001). Compensatory mechanisms active during the development of PBX1^{-/-} embryos could affect variety of intrinsic and extrinsic mechanisms involved in establishment of normal adult HSC pool, and could thus alter the inherent responsiveness of HSC to HOXB4. Although the antisense approach used in this study was clearly successful in producing major reduction in levels of PBX1 (see FIGS. 4 and 5), and was specific enough such that the levels of paralogs PBX2 and PBX3 remained unperturbed (FIG. 5), the possibility that the expression levels of other proteins might be affected cannot be ruled out. However, our ongoing studies indicate that co-overexpression of PBX1 reverses the repopulation advantage conferred to HSC by HOXB4, and thus strongly argue for a PBX1-dependent effect of our knock down strategy.

HOXB4 is capable of inducing a significant in vivo and ex vivo expansion of adult hematopoietic stem cells (Sauvageau, G., et al., Genes Dev. 9: 1753-1765, 1995; Thorsteinsdottir, U., et al., Blood. 94: 2605-2612, 1999; Antonchuk, J., et al., Exp. Hematol. 29: 1125-1134, 2001; Antonchuk, J., et al., Cell. 109: 39-45, 2002). Results presented in this disclosure show that decrease in levels of endogenous PBX1 can further increase the competitive proliferation potential of HOXB4-transduced HSC by ˜20-fold. We show that the proliferative effect of PBX1^{K.D.} is mainly restricted to the most primitive HOXB4-transduced hematopoietic cells, that these cells expand up to but not beyond the normal HSC levels, and retain their capacity to generate progeny committed to all hematopoietic lineages. Our observations also show that the presence of more competitive HOXB4-PBX1^{K.D}-transduced populations decreases the proliferation activity of HOXB4 cells, suggesting an existence of extrinsic mechanism(s) that control the extent of HOXB4-induced stem cell expansion.

Followings are the material and methods used in this investigation.

Animals: (C57BI/6J×C3H/HeJ)F1 and (C57BI/6Ly-Pep3b×C3H/HeJ)F1 mice were bred at specific pathogen free (SPF) animal facility of the Clinical Research Institute of Montreal. All animals were housed in ventilated cages and provided with sterilized food and acidified water.

Retroviral vectors: Generation of the MSCV-IRES-GFP and MSCV-HOXB4-IRES-GFP vectors were described before (Antonchuk, J., et al., Exp. Hematol. 29: 1125-1134, 2001). To generate MSCV-a/s PBX1b-PGK-YFP vector, a 1.4 kb fragment encoding PBX1b ORF was blunted and subcloned into Hpa I site of MSCV-PGK-YFP (EK, EMBO 2000).

Retroviral Infection and Transplantation of Primary Bone Marrow Cells

Generation of retrovirus producing GP+E-86 cells, and single or double infections of bone marrow cells were performed as described previously (Kroon, E., et al., EMBO J. 20: 350-361, 2001). Control competitor bone marrow cells were co-cultured with non-transduced GP+E-86 cells, but were otherwise treated exactly like cells subjected to retroviral infection. 1 day after recovery from co-culture with retroviral producers, the proportions of transduced (GFP⁺, YFP⁺, or GFP⁺/YFP⁺) cells were determined by flow cytometry using MoFlo (Cytomation, Fort Collins, Colo.). Transplantation inocula comprising 10% GFP⁺ or YFP⁺ cells were prepared by diluting the infected cell populations with non-transduced competitors. Doubly transduced (HOXB4-GFP, and a/s PBX1-YFP, and HOXB4-GFP+a/s PBX1-YFP) cell populations were diluted with competitors such that the proportion of GFP⁺/YFP⁺ cells always represented 10% of the transplants. 12- to 16-week-old recipient mice were irradiated (850 cGy, 160 cGy/min, ¹³⁷Cs γ source, J. L. Shepherd, CA), and transplanted with 2.5×10⁵ cells, together with 1×10⁵ freshly isolated bone marrow cells to provide for transient radioprotection.

In Vitro Proliferation of Primary Bone Marrow Cells

To determine the in vitro competitive proliferation potential of the transduced cells, cultures comprising varying proportions of GFP⁺, and YFP⁺, and GFP⁺/YFP⁺ bone marrow cell populations were initiated at 5×10⁴ cells/mL in media supplemented with 15% FCS and 10 ng/mL of IL-3 one day after recovery from co-culture with retroviral producers. In 3 day-intervals, viable (trypan blue negative) cells were counted and diluted with fresh media such that cell density was maintained between 5×10⁴ and 5×10⁵ cells/mL. At the same time points, the relative contents of GFP⁺, and YFP⁺, and GFP⁺/YFP⁺ cells were determined by flow cytometry. To is determine the in vitro proliferation potential of the transduced myeloid CFC, GFP⁺, or YFP⁺, or GFP⁺/YFP⁺ cells were sorted one day after recovery from co-culture with retroviral producers, and liquid cultures were initiated and maintained as described above. After 6 and 12 days of growth, suitable aliquots of cultures were plated in methycelullose containing 10 ng/mL of IL-3, 10 ng/mL of IL-6, 50 ng/mL of SCF, and 5 U/mL of Epo (hereafter, standard methylcellulose). Colonies were scored on day 10. Methylcellulose and cytokines in a form of COS cell-derived supernatants used for these experiments were prepared and quantitated at IRCM. All other media components were purchased from GIBCO/Invitrogen Corp. (Burlington, ON, Canada).

Flow Cytometry

Cells isolated from bone marrow, spleen and thymus of transplantation chimeras were resuspended in PBS with 2% FCS and allophycocyanin (APC)-conjugated antibodies recognizing Mac-1, or B-200, or CD4 and CD8 cell surface markers (Pharmingen, Mississauga, ON). Following 30 min incubation on ice, cells were washed 2× with PBS with 2% FCS, and fractions of GFP⁺, YFP⁺, and GFP⁺/YFP⁺ cells that express a given cell surface antigen were determined by flow cytometry using MoFlo (Cytomation, Fort Collins, Colo.). Data were analyzed using Summit V3.1 software (Cytomation, Fort Collins, Colo.). Ly 5.1⁺ peripheral blood derived MNC were identified using FITC-conjugated anti-Ly 5.1 antibody (Pharmingen Mississauga, ON).

Competitive Repopulation and Cru Assays

To determine contributions of the transplanted transduced (GFP⁺, YFP⁺, and GFP⁺/YFP⁺) HSC to hematopoietic reconstitution at various intervals post transplantation, ˜50 μL of blood obtained from tail vein were incubated with excess of ammonium chloride (StemCell Technologies) to lyse erythrocytes, and the proportions of GFP⁺, YFP⁺, and GFP⁺/YFP⁺ PBL were determined by flow cytometry. Mice that had greater than 2% GFP⁺, or YFP⁺, or GFP⁺/YFP⁺ cells in both, myeloid (SSC^{hi}FSC^{low}) and lymphoid (SSC^{low}FSC^{hi}) subpopulations were considered to be repopulated with at least 1 transduced HSC. Fidelity of discrimination between myeloid and lymphoid cells was verified using cell surface staining to detect lineage specific markers (Mac-1 vs B-220). HSC numbers in primary recipients were evaluated using a limiting dilution transplantation-based assay (CRU assay), that detects cells with competitive, long-term lympho-myeloid repopulation capacity (Szilvassy S J, PNAS 1990). Briefly, primary Hoxb-GFP⁺, or Hoxb-GFP⁺ plus a/s PBX1-YFP⁺ plus a/s PBX1-YFP⁺+HOXB4-GFP⁺ recipients were sacrificed at 16 or 32 weeks post transplantation, and their bone marrow cells were injected into lethally irradiated secondary recipients at varying dilutions (5×10³-1×10⁶ cells/recipient, 5-10 recipients/dilution) along with 1×10⁵ freshly isolated helper bone marrow cells. The level of lymphoid and myeloid repopulation with donor-derived GFP⁺, or YFP⁺, or GFP⁺/YFP⁺ cells in secondary recipients was evaluated at 16 weeks post transplantation by flow cytometric analysis of PBL. CRU frequencies were calculated by applying Poisson statistics to the proportion of negative recipients at different dilutions using Limit Dilution Analysis software (StemCell Technologies). The same type of analysis was applied to autoradiograms generated by Southern blot analysis of proviral integrations in DNA isolated from bone marrow and thymus of recipients, in which the presence of HOXB4-GFP and/or a/s PBX1-YFP provirus identified the reconstituted recipients. To determine CRU frequencies in bone marrow populations introduced in co-culture with retroviral producers, bone marrow from 5-FU treated Ly 5.1 mice ([C57BI/6Ly-Pep3b×C3H/HeJ]F1) was cultured for 2 days in media supplemented with IL-3 (6 ng/mL), IL-6 (10 ng/mL), and SCF (100 ng/mL), and transplanted at various dilutions (5×10³-1×10⁶ cells/recipient, 5-10 recipients/dilution) in lethally irradiated Ly 5.2 mice (C57BI/6J×C3H/HeJ)F1. Contribution of transplant derived Ly 5.1⁺ cells to reconstitution of secondary recipients was estimated by flow cytometry at 16 weeks post transplantation, and CRU frequencies in test sample were determined as described above.

CFC Assays and In Vitro Expansion of Myeloid Clones

The frequencies of myeloid and preB clonogenic progenitors in bone marrow and spleen cell populations of primary and secondary recipients were determined as described (Thorsteinsdottir, U., et al., Blood. 99: 121-129, 2002). To generate clonal myeloid cell populations, individual well isolated multilineage mixed colonies were transferred from methylcellulose to liquid cultures in IMDM, supplemented with 15% FSC, IL-3 (10 ng/mL), IL-6 (10 ng/mL), IL-11 (100 ng/mL), SCF (10 ng/mL), and 10⁻⁵M β-mercaptoethanol. Clonal populations were then expanded for 10-14 days for total cell yields of ≧10⁷ cells/culture.

Southern and Western Blot Analysis

High molecular weight DNA from bone marrow, spleen and thymus of transplantation chimeras and from myeloid clones was digested with Kpn I, which cuts in the long terminal repeats (LTRs) to release 4.4 kb a/s PBX1-YFP proviral genome, and in LTRs and IRES of HOXB4-GFP provirus to separate the 2.3 kb HOXB4- and the 1.7 kb GFP-comprising fragments of HOXB4-GFP provirus. To detect unique HOXB4 proviral integrations, DNA was cut with EcoR I and Hind III, which release HOXB4 cDNA from HOXB4-GFP provirus, and excise the 3 kb a/s PBX1-PGK-YFP cassette from a/s Pbx-YFP provirus. Southern blot analyses were performed using standard techniques. Probes used were 600 bp (nt 930-1525 of the ORF) PVU II fragment of PBX1, 490 bp Sal I-Pml I fragment of HOXB4 cDNA, and 730 bp YFP cDNA. Preparation of total cell lysates and Western blot analyses were performed as described (Krosl, J. and Sauvageau, G., Oncogene. 19: 5134-5141% 2000). Primary antibodies used were rat anti HOXB4 (Gould, A., et al., Genes Dev. 11: 900-913, 1997, and Developmental Studies Hybridoma Bank, University of Iowa), rabbit anti PBX1, rabbit anti c-Jun, and horseradish peroxidase-conjugated anti rat and anti rabbit antibodies (Santa Cruz Biotech., Santa Cruz, Calif.).

cDNA Generation, Amplification and Analysis

Reverse transcription and amplification of total RNA isolated from purified bone marrow subpopulations were performed as described (JL, G&D 1999). Probes used were 600 bp Pvu II fragment of PBX1 described above, 540 bp (nt 670-1210 of the ORF) Pvu II fragment of Pbx 2, 540 bp (nt 400-940) Pvu II fragment of Pbx 3, and actin cDNA. Intensities of autoradiographic signals identifying PBX gene expression were evaluated using Alpha Imager™ 2000 Documentation and Analysis system (Alphalnnotech, San Leandro, Calif.).

While the invention has been described in connection with specific a specific example, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A stem cell expansion factor comprising a blocker which reduces expression level of at least one gene normally limiting HOX-induced expansion of stem cells, whereby reducing expression level of said gene enhances expansion of stem cells containing a HOX peptide.

2. The stem cell expansion factor of claim 1, wherein said blocker is selected from the group consisting of an antisense, an antibody, a SiRNA, a peptide and a chemical compound.

3. The stem cell expansion factor of claim 1, wherein said gene is a PBX gene.

4. The stem cell expansion factor of claim 3, wherein said blocker is a nucleic acid sequence blocking PBX expression.

5. The stem cell expansion factor of claim 4, wherein said blocker is an antisense DNA to PBX1.

6. The stem cell expansion factor of claim 1, wherein said blocker is a PBX1 expression blocker.

7. The stem cell expansion factor of claim 1, wherein said stem cells are hematopoietic stem cells.

8. The stem cell expansion factor of claim 7, wherein said hematopoietic stem cells are human or mouse hematopoietic stem cells.

9. A nucleic acid construct for enhancing stem cells expansion, said construct comprising a first nucleic acid sequence for expression of a HOX peptide, wherein said peptide being able to cross a cell membrane, and a second nucleic acid sequence blocking expression of at least one gene normally limiting HOX-induced expansion of stem cells, whereby reducing expression level of said gene in the presence of a HOX peptide enhances expansion of stem cells.

10. The construct of claim 9, wherein said gene is a PBX gene.

11. The construct of claim 9, wherein said HOX peptide is a HOXB4 peptide.

12. The construct of claim 9, wherein said stem cells are hematopoietic stem cells.

13. The construct of claim 12, wherein said hematopoietic stem cells are human or mouse hematopoietic stem cells.

14. The construct of claim 10, wherein said second nucleic acid sequence blocking PBX expression is an antisense DNA to PBX1.

15. A composition for enhancing expansion of stem cells comprising an amino acid sequence having the activity of a HOX peptide, wherein said peptide being able to cross a cell membrane, and a blocker which reduces expression level of at least one gene normally limiting HOX-induced expansion of stem cells, whereby reducing expression level of said gene in the presence of a HOX peptide enhances expansion of stem cells.

16. The composition of claim 15, wherein said gene is a PBX gene.

17. The composition according to claim 15, wherein said amino acid sequence consists of a HOXB4 peptide.

18. The composition according to claim 15, wherein said amino acid sequence comprises an HIV-derived peptide able to cross a cell membrane.

19. The composition according to claim 18, wherein said HIV-derived peptide consists of a NH2-terminal protein transduction domain (PTD) from a transactivating protein.

20. The composition according to claim 15, wherein said stem cells are hematopoietic stem cells.

21. The composition according to claim 20, wherein said hematopoietic stem cells are human or mouse hematopoietic stem cells.

22. The composition according to claim 16, wherein said blocker is a nucleic acid sequence blocking PBX expression.

23. The composition according to claim 22, wherein said blocker is an antisense DNA to PBX1.

24. A composition for enhancing expansion of stem cells comprising a nucleic acid sequence for over-expression of a HOX peptide, and a blocker which reduces expression level of at least one gene normally limiting HOX-induced expansion of stem cells, whereby reducing expression level of said gene in the presence of a overexpressed HOX peptide enhances expansion of stem cells.

25. The composition of claim 24, wherein said gene is a PBX gene.

26. The composition according to claim 24, wherein said HOX peptide is a HOXB4 peptide.

27. The composition according to claim 24, wherein said stem cells are hematopoietic stem cells.

28. The composition according to claim 27, wherein said hematopoietic stem cells are human or mouse hematopoietic stem cells.

29. The composition according to claim 24, wherein said blocker is a nucleic acid sequence blocking PBX expression.

30. The composition according to claim 29, wherein said blocker is an antisense DNA to PBX1.

31. A method for enhancing expansion of stem cells, which comprises treating stem cells with an effective amount of a factor as defined in claim 1, or an effective amount of a composition as defined in claim 15 for a time sufficient to allow expansion of said stem cells.

32. The method of claim 31, wherein said HOX peptide is a HOXB4 peptide and said gene is PBX.

33. The method of claim 31, further comprising a step of treating said stem cell with an amino acid sequence having the activity of a HOX peptide encoded by a HOX nucleotide sequence.

34. The method of claim 33, wherein said amino acid sequence consists of a HOXB4 peptide.

35. The method of claim 33, wherein said amino acid sequence comprises an HIV-derived peptide able to cross a cell membrane.

36. The method of claim 35, wherein said HIV-derived peptide consists of a NH2-terminal protein transduction domain (PTD) from a transactivating protein.

37. The method of claim 31, wherein said stem cells are hematopoietic stem cells.

38. The method of claim 37, wherein said hematopoietic stem cells are human or mouse hematopoietic stem cells.

39. The method of anyone of claims 31 to 38, wherein said stem cells are treated in vitro, in vivo or ex vivo.

40. (canceled)

41. A method for restoring hematopoietic capability of a patient, which comprises administering a therapeutical effective amount of a factor as defined in claim 1.

42. A method for restoring hematopoietic capability of a patient, which comprises administering a therapeutical effective amount of a construct as defined in claim 9.

43. A method for restoring hematopoietic capability of a patient, which comprises administering a therapeutical effective amount of a composition as defined in claim 15.

44. A method for enhancing expansion of stem cells, which comprises treating stem cells with an effective amount of a factor as defined in claim 15 for a time sufficient to allow expansion of said stem cells.
